(19) United States
(12) Patent Application Publication
(10) Pub. No.: US 2025/0005904 A1
(43) Pub. Date: Jan. 2, 2025

(54) MEDICAL ASSISTANCE SYSTEM AND IMAGE DISPLAY METHOD

(71) Applicant: OLYMPUS MEDICAL SYSTEMS CORP., Tokyo (JP)

(72) Inventors: Shiho MIYAUCHI, Tokyo (JP); Kazuya WATANABE, Tokyo (JP); Takashi NAGATA, Tokyo (JP); Ryo OGUMA, Tokyo (JP); Satomi KOBAYASHI, Tokyo (JP); Kazuya FURUHO, Tokyo (JP); Isao TATESHITA, Tokyo (JP)

(73) Assignee: OLYMPUS MEDICAL SYSTEMS CORP., Tokyo (JP)

(21) Appl. No.: 18/885,008

(22) Filed: Sep. 13, 2024

Related U.S. Application Data
(63) Continuation of application No. PCT/JP2022/012652, filed on Mar. 18, 2022.

(51) Int. Cl. G06V 10/764; G06F 3/0482; G06F 3/14; G06T 7/90; G06V 10/60

(52) U.S. Cl. CPC G06V 10/764; G06F 3/0482; G06F 3/14; G06T 7/90; G06V 10/60

(57) ABSTRACT

A processing unit classifies multiple images taken inside a subject's body into multiple groups based on a certain criterion. The processing unit distinguishably displays images in the multiple groups.

### CROSS-REFERENCE TO RELATED APPLICATIONS

This application is based upon and claims the benefit of priority from the International Application No. PCT/JP2022/012652, filed on Mar. 18, 2022, the entire contents of which are incorporated.

### BACKGROUND

### 1. Technical Field

The present disclosure relates to a medical assistance system and an image display method for displaying images taken inside a subject's body.

### 2. Description of the Related Art

In endoscopy, a doctor observes images taken by an endoscope inserted into a subject's body and displayed on a display device. When an image showing a lesion, such as a bleeding area, is displayed, the doctor operates the release switch of the endoscope to capture (save) the endoscopic image. Since the doctor observes (interprets) the captured images again after the examination, the more images captured, the longer the time required for the image interpretation.

JP-A-2006-320650 discloses an image display device that sequentially displays a series of images. The image display device disclosed in JP-A-2006-320650 classifies images into multiple image groups according to the degree of correlation between images, extracts a feature image having a feature image area as a representative image in each image group, and sequentially displays the representative images of the multiple image groups.

### SUMMARY

A medical assistance system according to one embodiment of the present disclosure includes a processor including hardware, and the processor classifies multiple images taken inside a subject's body into multiple groups based on a predetermined criterion and displays the multiple groups distinguishable from each other.

An image display method according to another embodiment of the present disclosure includes: classifying multiple images taken inside a subject's body into multiple groups based on a predetermined criterion; and displaying the multiple groups distinguishable from each other.

Optional combinations of the aforementioned constituting elements, and implementation of the present disclosure in the form of methods, apparatuses, systems, recording media, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram that shows a configuration of a medical assistance system according to an embodiment;

FIG. 2 is a diagram that shows functional blocks of a server device;

FIG. 3 is a diagram that shows functional blocks of an information processing device;

FIG. 4 is a diagram that shows an example of a report creation screen;

FIG. 5 is a diagram that shows multiple endoscopic images;

FIG. 6 is a diagram that shows another example of multiple endoscopic images;

FIG. 7 is a diagram that shows examples of a bleeding source candidate image;

FIG. 8 is a diagram that shows bleeding areas in bleeding images;

FIG. 9 is a diagram that shows an example of a list screen for bleeding images;

FIG. 10 is a diagram that shows an example of a list screen for bleeding source candidate images;

FIG. 11 is a diagram that shows another example of a list screen for bleeding images;

FIG. 12 is a diagram that shows an example of a group selection screen;

FIG. 13 is a diagram that shows an example of a playback screen for an endoscopic image; and

FIG. 14 is a diagram that shows an example of a screen displayed during automatic sequential display.

### DETAILED DESCRIPTION

The disclosure will now be described by reference to the preferred embodiments. This does not intend to limit the scope of the present disclosure, but to exemplify the disclosure.

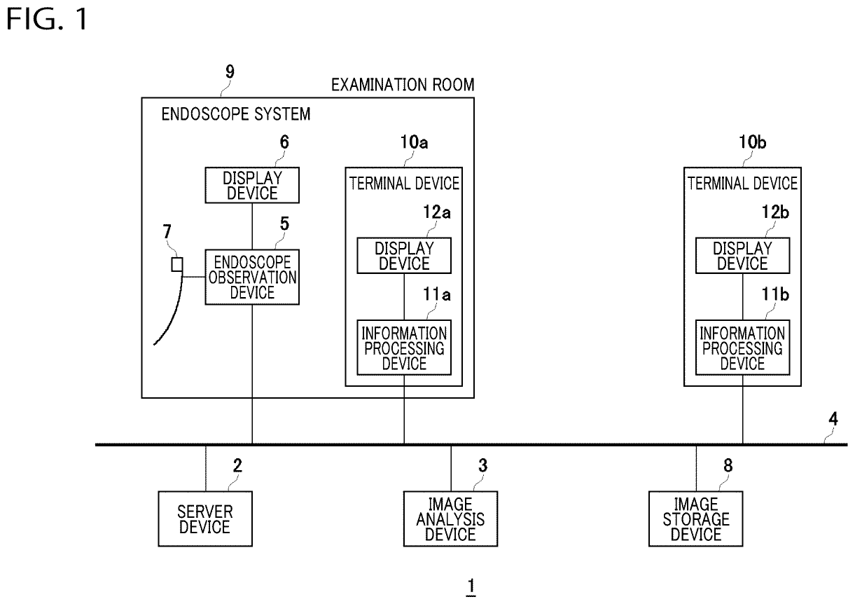

FIG. 1 shows a configuration of a medical assistance system 1 according to an embodiment. The medical assistance system 1 is installed in a medical facility, such as a hospital where endoscopy is performed. In the medical assistance system 1, a server device 2, an image analysis device 3, an image storage device 8, an endoscope system 9, and a terminal device 10b are communicatively connected via a network 4, such as a local area network (LAN). The endoscope system 9 is installed in an examination room and includes an endoscope observation device 5 and a terminal device 10a. In the medical assistance system 1, the server device 2, image analysis device 3, and image storage device 8 may be provided as a cloud server, for example, outside the medical facility.

To the endoscope observation device 5, an endoscope 7, which is inserted into a patient's digestive tract, is connected. The endoscope 7 includes a light guide for transmitting illumination light supplied from the endoscope observation device 5 to illuminate the inside of the digestive tract. The endoscope 7 includes, at the tip thereof, an illumination window through which illumination light transmitted by the light guide is emitted to a biological tissue, and an image shooting unit that shoots an image of a biological tissue with a predetermined period and outputs an imaging signal to the endoscope observation device 5. The image shooting unit includes a solid-state image sensor (e.g., a CCD image sensor or a CMOS image sensor) that converts the incident light into an electrical signal.

The endoscope observation device 5 performs image processing on the imaging signal photoelectrically converted by the solid-state image sensor of the endoscope 7 to generate an endoscopic image and displays the endoscopic image in real time on a display device 6. The endoscope observation device 5 may have a function to perform special image processing for highlighting or the like, in addition to normal image processing including A/D conversion and noise removal. The imaging frame rate of the endoscope 7 may preferably be 30 fps or higher and may be 60 fps. The endoscope observation device 5 generates an endoscopic image with the same period as the imaging frame rate. The endoscope observation device 5 may be constituted by one or more processors with dedicated hardware or may also be constituted by one or more processors with general-purpose hardware. The endoscope 7 of the embodiment is a flexible endoscope and includes a forceps channel into which an endoscopic instrument is inserted. By inserting a biopsy forceps into the forceps channel and operating the biopsy forceps thus inserted, the doctor can perform a biopsy during endoscopy and obtain a portion of a lesional tissue.

The doctor operates the endoscope 7 according to the examination procedure and observes an endoscopic image displayed on the display device 6. In lower endoscopy, the doctor usually inserts the endoscope 7 for lower examination from the anus to the end of the ileum and observes, while withdrawing the endoscope 7, the end of the ileum and the large intestine in sequence. Also, in upper endoscopy, the doctor inserts the endoscope 7 for upper examination from the mouth to the duodenum and observes, while withdrawing the endoscope 7, the duodenum, stomach, and esophagus in sequence. In upper endoscopy, the doctor may observe the esophagus, stomach, and duodenum in sequence while inserting the endoscope 7.

When a biological tissue to be captured is displayed on the display device 6, the doctor operates the release switch of the endoscope 7. The endoscope observation device 5 captures an endoscopic image at the timing when the release switch is operated and then transmits the endoscopic image thus captured, together with information identifying the endoscopic image (an image ID), to the image storage device 8. The endoscope observation device 5 may assign an image ID including a serial number to each of endoscopic images in the order in which they were captured. Also, the endoscope observation device 5 may transmit multiple captured endoscopic images collectively to the image storage device 8 after the examination is finished. The image storage device 8 records an endoscopic image transmitted from the endoscope observation device 5, in relation to an examination ID that identifies the endoscopic examination.

In the embodiment, “image shooting” means an operation in which the solid-state image sensor of the endoscope 7 converts the incident light into an electrical signal. The “image shooting” may include the operation until the endoscope observation device 5 generates an endoscopic image from the converted electrical signal and may further include the operation until the endoscopic image is displayed on the display device 6. In the embodiment, “capturing” means the operation of acquiring an endoscopic image generated by the endoscope observation device 5. The “capturing” may also include the operation of saving (recording) the endoscopic image thus acquired. In the embodiment, a shot endoscopic image is captured when the doctor operates the release switch; however, the shot endoscopic image may be automatically captured, regardless of the operation of the release switch.

The terminal device 10a includes an information processing device 11a and a display device 12a and is installed in the examination room. The terminal device 10a may be used by a doctor, nurse, or others to check, in real time, information related to the biological tissue of which images are shot during endoscopy.

The terminal device 10b includes an information processing device 11b and a display device 12b and is installed in a room other than the examination room. The terminal device 10b is used by a doctor to prepare a report of an endoscopic examination. Each of the terminal devices 10a and 10b in a medical facility may be constituted by one or more processors with general-purpose hardware.

In the medical assistance system 1 of the embodiment, the endoscope observation device 5 displays an endoscopic image in real time on the display device 6 and also provides the endoscopic image together with meta-information of the image to the image analysis device 3 in real time. The meta-information includes at least a frame number and shooting time information of the image, and the frame number may be information indicating what number frame it is since the endoscope 7 started image shooting.

The image analysis device 3 is an electronic computer (a computer) that analyzes an endoscopic image, detects a lesion in the endoscopic image, and performs qualitative diagnosis of the detected lesion. The image analysis device 3 may be a computer-aided diagnosis (CAD) system with an artificial intelligence (AI) diagnostic function. The image analysis device 3 may be constituted by one or more processors with dedicated hardware or may also be constituted by one or more processors with general-purpose hardware.

The image analysis device 3 uses a learned model created by machine learning using, as teacher data, endoscopic images for learning, information indicating organs and regions included in the endoscopic images, and information regarding lesion areas included in the endoscopic images. Annotation work on the endoscopic images is performed by an annotator having expertise, such as a doctor. For the machine learning, a CNN, an RNN, or LSTM, which is a type of deep learning, may be used. When an endoscopic image is input, the learned model outputs information indicating an organ shown in the image, information indicating a region shown in the image, and information regarding a lesion shown in the image (lesion information). The lesion information output by the image analysis device 3 includes at least lesion presence/absence information that indicates whether or not a lesion is included (or shown) in the endoscopic image. When a lesion is included, the lesion information may include information indicating the size of the lesion, information indicating the location of the outline of the lesion, information indicating the shape of the lesion, information indicating the depth of lesion invasion, and a qualitative diagnosis result for the lesion. The qualitative diagnosis result for the lesion includes information indicating the type of the lesion and may include information indicating, for example, being in a bleeding state.

During an endoscopic examination, the image analysis device 3 is provided with endoscopic images from the endoscope observation device 5 in real time and outputs, for each endoscopic image, the information indicating an organ, the information indicating a region, and the lesion information. Hereinafter, the information indicating an organ, the information indicating a region, and the lesion information output for each endoscopic image will be collectively referred to as “image analysis information”. The image analysis device 3 may generate color information (an averaged color value) obtained by averaging the pixel values of an endoscopic image, and such color information may be included in the image analysis information.

When the user operates the release switch (capturing operation), the endoscope observation device 5 provides, to the image analysis device 3, the frame number, the shooting time, and the image ID of the captured endoscopic image, together with information indicating that the capturing operation has been performed (capturing operation information). Upon acquisition of the capturing operation information, the image analysis device 3 provides, to the server device 2, the image ID, the frame number, the shooting time information, and the image analysis information of the provided frame number, together with the examination ID. The image ID, frame number, shooting time information, and image analysis information as mentioned herein constitute “additional information” that indicates features and properties of the endoscopic image. Upon acquisition of the capturing operation information, the image analysis device 3 transmits the additional information together with the examination ID to the server device 2, and the server device 2 records the additional information in relation to the examination ID.

When the endoscopic examination is finished, the user operates an examination finish button of the endoscope observation device 5. Information on the operation of the examination finish button is provided to the server device 2 and the image analysis device 3, and the server device 2 and the image analysis device 3 recognize the end of the endoscopic examination.

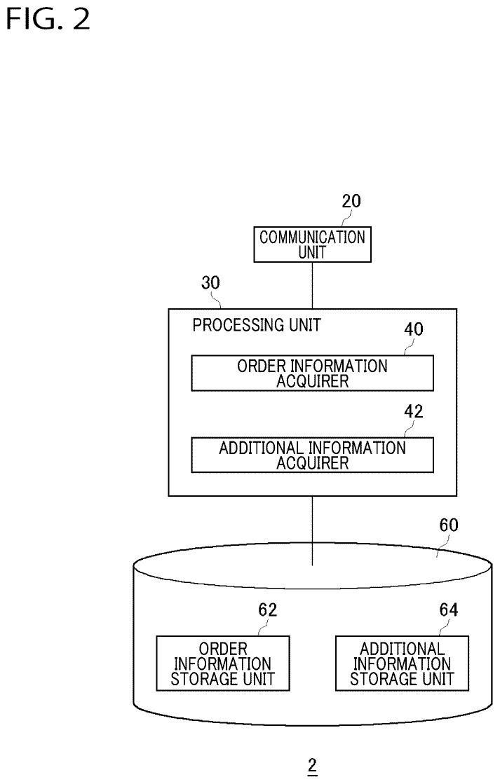

FIG. 2 shows functional blocks of the server device 2. The server device 2 includes a communication unit 20, a processing unit 30, and a storage device 60. The communication unit 20 transmits and receives information, such as data and instructions, to and from the image analysis device 3, the endoscope observation device 5, the image storage device 8, the terminal device 10a, and the terminal device 10b, via the network 4. The processing unit 30 includes an order information acquirer 40 and an additional information acquirer 42. The storage device 60 includes an order information storage unit 62 and an additional information storage unit 64.

The server device 2 includes a computer, and the computer executes programs to implement various functions shown in FIG. 2. The computer includes, as hardware, a memory into which programs are loaded, one or more processors that execute the loaded programs, an auxiliary storage device, and other LSIs. Each processor is constituted by multiple electronic circuits including a semiconductor integrated circuit and an LSI. The multiple electronic circuits may be mounted on a single chip or may be mounted on multiple chips. The functional blocks shown in FIG. 2 are implemented by coordination of hardware and software. Therefore, it will be understood by those skilled in the art that these functional blocks may be implemented in a variety of forms by hardware only, software only, or a combination thereof.

The order information acquirer 40 acquires order information for an endoscopic examination from a hospital information system. For example, before the start of examination work for a day at a medical facility, the order information acquirer 40 acquires the order information for the day from the hospital information system and stores the order information in the order information storage unit 62. Before the start of an examination, the endoscope observation device 5 or the information processing device 11a may read the order information of the examination to be performed from the order information storage unit 62 and display the order information on a display device.

The additional information acquirer 42 acquires, from the image analysis device 3, the examination ID and the additional information of an endoscopic image and stores the additional information in relation to the examination ID in the additional information storage unit 64. The additional information of an endoscopic image includes the image ID, frame number, shooting time information, and image analysis information.

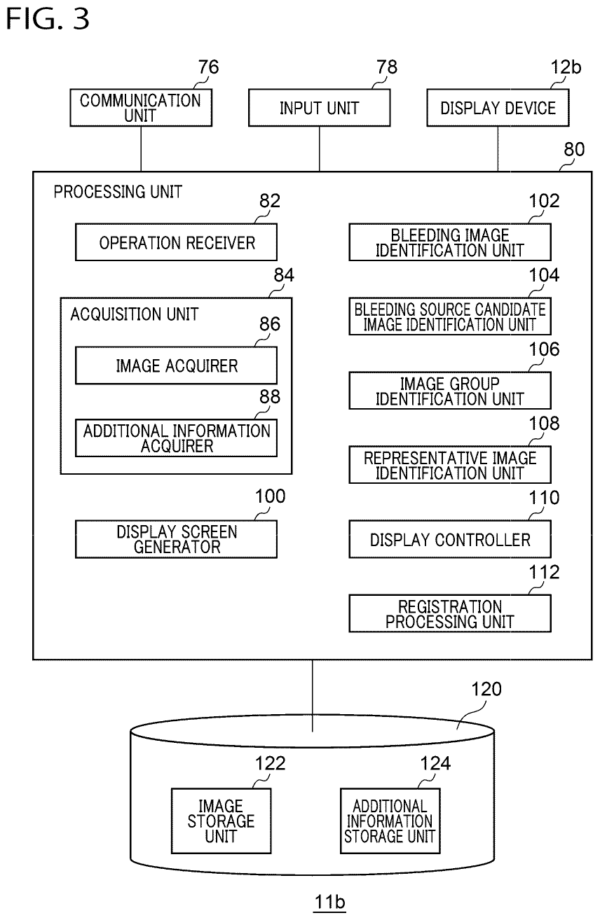

FIG. 3 shows functional blocks of the information processing device 11b. The information processing device 11b has a function to support operations for creating examination reports and includes a communication unit 76, an input unit 78, a processing unit 80, and a storage device 120. The communication unit 76 transmits and receives information, such as data and instructions, to and from the server device 2, the image analysis device 3, the endoscope observation device 5, the image storage device 8, and the terminal device 10a, via the network 4. The processing unit 80 includes an operation receiver 82, an acquisition unit 84, a display screen generator 100, a bleeding image identification unit 102, a bleeding source candidate image identification unit 104, an image group identification unit 106, a representative image identification unit 108, a display controller 110, and a registration processing unit 112. The acquisition unit 84 includes an image acquirer 86 and an additional information acquirer 88. The storage device 120 includes an image storage unit 122 and an additional information storage unit 124.

The information processing device 11b includes a computer, and the computer executes programs to implement various functions shown in FIG. 3. The computer includes, as hardware, a memory into which programs are loaded, one or more processors that execute the loaded programs, an auxiliary storage device, and other LSIs. Each processor is constituted by multiple electronic circuits including a semiconductor integrated circuit and an LSI. The multiple electronic circuits may be mounted on a single chip or may be mounted on multiple chips. The functional blocks shown in FIG. 3 are implemented by coordination of hardware and software. Therefore, it will be understood by those skilled in the art that these functional blocks may be implemented in a variety of forms by hardware only, software only, or a combination thereof.

After an endoscopic examination is finished, the user as a doctor enters a user ID and a password into the information processing device 11b to log in. When the user logs in, an application for creating an examination report is started, and a list of performed examinations is displayed on the display device 12b. In this list of performed examinations, examination information such as the patient's name, patient ID, examination date and time, and examination item are listed, and the user selects an examination of which the report is to be created, by operating the input unit 78, which may be a mouse, a keyboard, and the like. When the operation receiver 82 receives an examination selection operation, the image acquirer 86 acquires, from the image storage device 8, multiple endoscopic images related to the examination ID of the examination selected by the user and stores the endoscopic images in the image storage unit 122. Also, the additional information acquirer 88 acquires, from the server device 2, the additional information related to the examination ID of the examination selected by the user and stores the additional information in the additional information storage unit 124. The display screen generator 100 generates a report creation screen and displays it on the display device 12b.

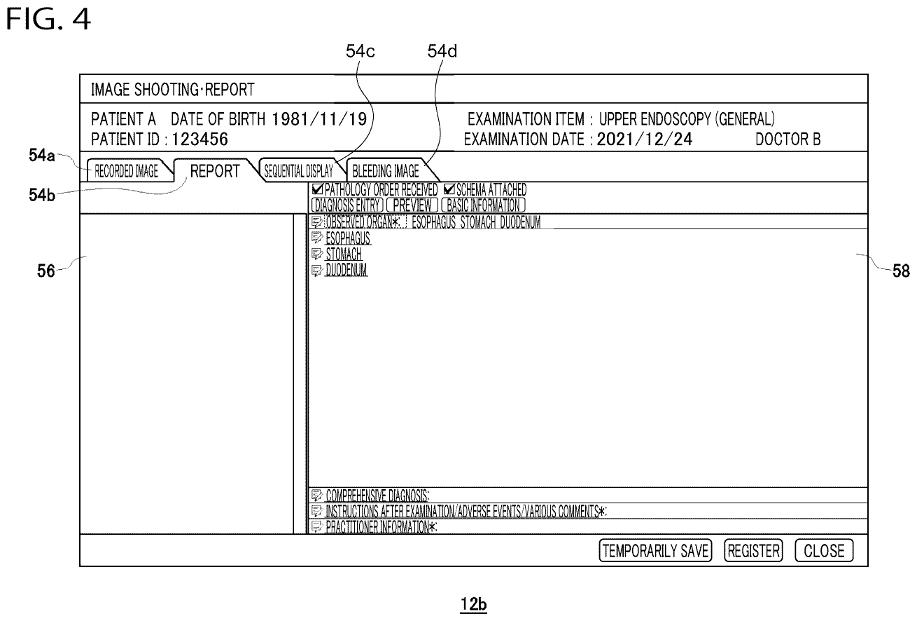

FIG. 4 shows an example of a report creation screen into which an examination result is entered. The report creation screen is displayed on the display device 12b with a report tab 54 selected. In an upper part of the screen, the patient's name, patient ID, date of birth, examination item, examination date, and information on the doctor who performed the examination are displayed. Such information is included in examination order information and may be acquired from the server device 2.

The report creation screen is constituted by two areas. In the left area, an attached image display area 56 is arranged in which an attached endoscopic image is displayed, and, in the right area, an entry area 58 is arranged into which the user enters an examination result. In the entry area 58, areas are provided into which the diagnostic details of the “esophagus”, “stomach”, and “duodenum”, which are observation ranges in upper endoscopy, are entered. The entry area 58 may have a format in which multiple options for examination results are displayed and the user selects checkboxes to enter the diagnostic details. The entry area 58 may also have a free format in which the user freely enters text.

The attached image display area 56 is an area in which endoscopic images attached to the report are arranged and displayed. The user selects an endoscopic image to be attached to the report from a list screen for endoscopic images, a playback screen for endoscopic images, or a list screen for bleeding images. When the user selects a recorded image tab 54a, the display screen generator 100 generates a list screen in which multiple endoscopic images obtained in the examination are arranged and displays the list screen on the display device 12b. When the user selects a sequential display tab 54c, the display screen generator 100 generates a playback screen, on which multiple endoscopic images obtained in the examination are sequentially displayed in the forward direction following the shooting order thereof or in the reverse direction, and displays the playback screen on the display device 12b. When the user selects a bleeding image tab 54d, the display screen generator 100 generates a list screen, in which images that contain blood (hereinafter, referred to as “bleeding images”) among multiple endoscopic images obtained in the examination are arranged, and displays the list screen on the display device 12b.

The bleeding image identification unit 102 identifies multiple bleeding images from among multiple endoscopic images. When blood is shown in an endoscopic image, the image analysis device 3 generates, as the lesion information of the endoscopic image, a qualitative diagnosis result indicating being in a bleeding state. Accordingly, the bleeding image identification unit 102 may refer to the additional information stored in the additional information storage unit 124 to identify an endoscopic image (a bleeding image) for which the qualitative diagnosis result indicating being in a bleeding state has been generated. When the additional information is not used, the bleeding image identification unit 102 may identify a bleeding image based on at least one of the saturation, hue, or lightness of an endoscopic image. In specific, the bleeding image identification unit 102 may derive the degree of redness (saturation) of an endoscopic image by image processing and may judge the endoscopic image to be a bleeding image when the degree of redness thus derived exceeds a predetermined threshold.

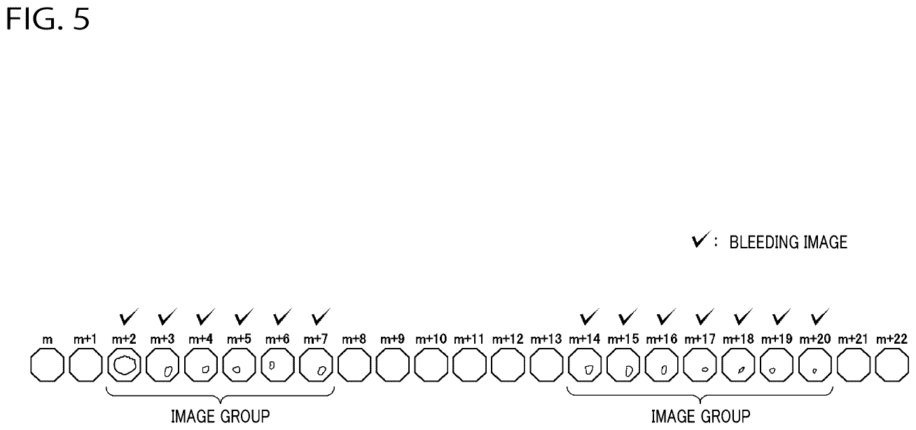

FIG. 5 shows an example in which multiple endoscopic images taken inside a subject's body are partially extracted. The octagons schematically represent the endoscopic images and are arranged from left to right in order of earliest to latest shooting time. In this example, an image (m) has the earliest shooting time, and an image (m+22) has the latest shooting time. The check marks shown above some images indicate that the images contain (show) bleeding. In the example shown in FIG. 5, images (m+2), (m+3), (m+4), (m+5), (m+6), (m+7), (m+14), (m+15), (m+16), (m+17), (m+18), (m+19), and (m+20) are bleeding images. The other images do not show bleeding.

When multiple bleeding images are consecutive in time, the image group identification unit 106 identifies the consecutive bleeding images as one image group. In this example, the image group identification unit 106 identifies the six temporally consecutive images from the image (m+2) to the image (m+7) as one image group and also identifies the seven temporally consecutive images from the image (m+14) to the image (m+20) as one image group.

The image group identification unit 106 may also identify multiple temporally consecutive images including at least two bleeding images as one image group, based on another condition.

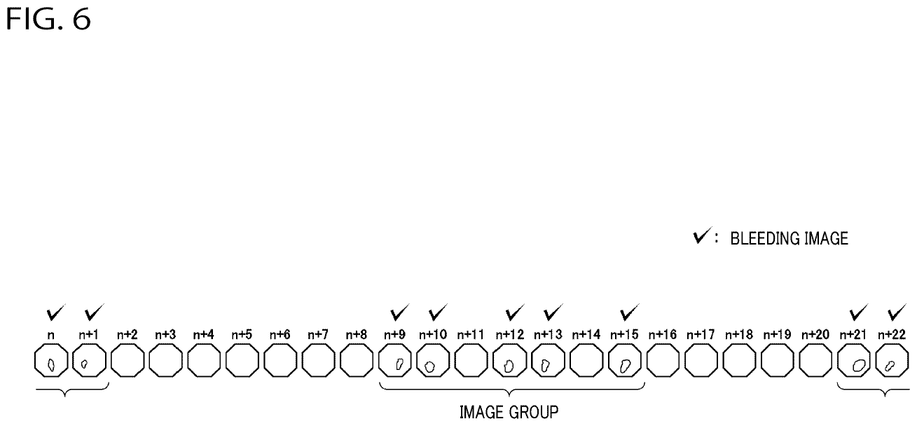

FIG. 6 shows another example in which multiple endoscopic images taken inside a subject's body are partially extracted. The octagons schematically represent the endoscopic images and are arranged from left to right in order of earliest to latest shooting time. In this example, an image (n) has the earliest shooting time, and an image (n+22) has the latest shooting time. The check marks shown above some images indicate that the images contain (show) bleeding. In the example shown in FIG. 6, images (n), (n+1), (n+9), (n+10), (n+12), (n+13), (n+15), (n+21), and (n+22) are bleeding images. The other images do not show bleeding.

The image group identification unit 106 may identify an image group including multiple bleeding images based on the distance between the shooting positions at which two bleeding images were respectively shot. The shooting position of a bleeding image may be the position of the tip of the endoscope 7 at the time when the bleeding image was shot or the position of a lesion. The shooting position of a bleeding image may be identified based on the region information included in the image analysis information or may be identified using other conventional techniques. When the distance between the shooting positions of two bleeding images exceeds a predetermined threshold Dth, the image group identification unit 106 does not include the two bleeding images in one image group; when the distance between the shooting positions of two bleeding images is less than or equal to the predetermined threshold Dth, on the other hand, the image group identification unit 106 includes the two bleeding images in one image group.

In the example shown in FIG. 6, the image group identification unit 106 investigates the distance between the shooting position of the image (n+1) and the shooting position of the image (n+9), which is the next bleeding image of the image (n+1), and, since the distance between the two shooting positions exceeds Dth, the image group identification unit 106 judges that the image (n+1) and the image (n+9) cannot be included in one image group.

Subsequently, the image group identification unit 106 investigates the distance between the shooting position of the image (n+9) and the shooting position of the image (n+10), which is the next bleeding image of the image (n+9), and, since the distance between the two shooting positions is less than or equal to Dth, the image group identification unit 106 judges that the image (n+9) and the image (n+10) can be included in one image group. The image group identification unit 106 then investigates the distance between the shooting position of the image (n+9) and the shooting position of the image (n+12), which is the next bleeding image of the image (n+10), and, since the distance between the two shooting positions is less than or equal to Dth, the image group identification unit 106 judges that the image (n+9) and the image (n+12) can be included in one image group. In the same way, the image group identification unit 106 also investigates the distance between the shooting position of the image (n+9) and the shooting position of the image (n+13) and the distance between the shooting position of the image (n+9) and the shooting position of the image (n+15) and, since each of the distances between two shooting positions is less than or equal to Dth, the image group identification unit 106 judges that the image (n+9), the image (n+13), and the image (n+15) can be included in one image group.

Furthermore, the image group identification unit 106 investigates the distance between the shooting position of the image (n+9) and the shooting position of the image (n+21), and, since the distance between the two shooting positions exceeds Dth, the image group identification unit 106 judges that the image (n+9) and the image (n+21) cannot be included in one image group. Based on the above judgment results, the image group identification unit 106 identifies the seven temporally consecutive images from the image (n+9) to the image (n+15) as one image group. Thus, the image group identification unit 106 may identify an image group including multiple bleeding images based on the distance between the shooting positions of two bleeding images.

The image group identification unit 106 may also identify an image group including multiple bleeding images based on the interval between the shooting times at which two bleeding images were respectively shot. The image group identification unit 106 refers to the additional information stored in the additional information storage unit 124 to identify the shooting times of bleeding images. Based on the interval between shooting times, the image group identification unit 106 identifies multiple temporally consecutive images including at least two bleeding images as one image group. When the interval between the shooting times of two bleeding images exceeds a predetermined threshold Tth, the image group identification unit 106 does not include the two bleeding images in one image group; when the interval between the shooting times of two bleeding images is less than or equal to the predetermined threshold Tth, on the other hand, the image group identification unit 106 includes the two bleeding images in one image group.

In the example shown in FIG. 6, the image group identification unit 106 investigates the interval between the shooting time of the image (n+1) and the shooting time of the image (n+9), which is the next bleeding image of the image (n+1), and, since the interval between the two shooting times exceeds Tth, the image group identification unit 106 judges that the image (n+1) and the image (n+9) cannot be included in one image group.

Subsequently, the image group identification unit 106 investigates the interval between the shooting time of the image (n+9) and the shooting time of the image (n+10), which is the next bleeding image of the image (n+9), and, since the interval between the two shooting times is less than or equal to Tth, the image group identification unit 106 judges that the image (n+9) and the image (n+10) can be included in one image group. The image group identification unit 106 then investigates the interval between the shooting time of the image (n+9) and the shooting time of the image (n+12), which is the next bleeding image of the image (n+10), and, since the interval between the two shooting times is less than or equal to Tth, the image group identification unit 106 judges that the image (n+9) and the image (n+12) can be included in one image group. In the same way, the image group identification unit 106 also investigates the interval between the shooting time of the image (n+9) and the shooting time of the image (n+13) and the interval between the shooting time of the image (n+9) and the shooting time of the image (n+15) and, since each of the intervals between two shooting times is less than or equal to Tth, the image group identification unit 106 judges that the image (n+9), the image (n+13), and the image (n+15) can be included in one image group.

Furthermore, the image group identification unit 106 investigates the interval between the shooting time of the image (n+9) and the shooting time of the image (n+21), and, since the interval between the two shooting times exceeds Tth, the image group identification unit 106 judges that the image (n+9) and the image (n+21) cannot be included in one image group. Based on the above judgment results, the image group identification unit 106 identifies the seven temporally consecutive images from the image (n+9) to the image (n+15) as one image group. Thus, the image group identification unit 106 may identify an image group including multiple bleeding images based on the interval between the shooting times of two bleeding images.

The image group identification unit 106 may also identify an image group including multiple bleeding images based on the number of other images shot between the shooting operations for two bleeding images. When the number of images, which are not bleeding images, included between two bleeding images exceeds a predetermined threshold Nth, the image group identification unit 106 does not include the two bleeding images in one image group; when the number of images, which are not bleeding images, included between two bleeding images is smaller than or equal to the predetermined threshold Nth, on the other hand, the image group identification unit 106 includes the two bleeding images in one image group.

For example, when the threshold Nth is set to four, since seven images are included between the image (n+1) and the image (n+9), the image group identification unit 106 judges that the image (n+1) and the image (n+9) cannot be included in one image group. Also, since five images are included between the image (n+15) and the image (n+21), the image group identification unit 106 judges that the image (n+15) and the image (n+21) cannot be included in one image group. Meanwhile, between adjacent bleeding images in the images (n+9), (n+10), (n+12), (n+13), and (n+15), more than four images, which are not bleeding images, are not included. Therefore, the image group identification unit 106 identifies the seven temporally consecutive images from the image (n+9) to the image (n+15) as one image group.

The image group thus identified is constituted by multiple images in which blood from the same bleeding section, i.e., the same bleeding source, is shown. Therefore, the image group identification unit 106 judges whether two bleeding images were shot in the same bleeding section or in different bleeding sections, based on at least one of the distance between the shooting positions of the two bleeding images, the interval between the shooting times of the two bleeding images, or the number of other images shot between the shooting operations for the two bleeding images.

In the embodiment, the bleeding source candidate image identification unit 104 identifies, among multiple bleeding images, an image that is highly likely to contain a bleeding source where blood is flowing out (hereinafter, referred to as a “bleeding source candidate image”).

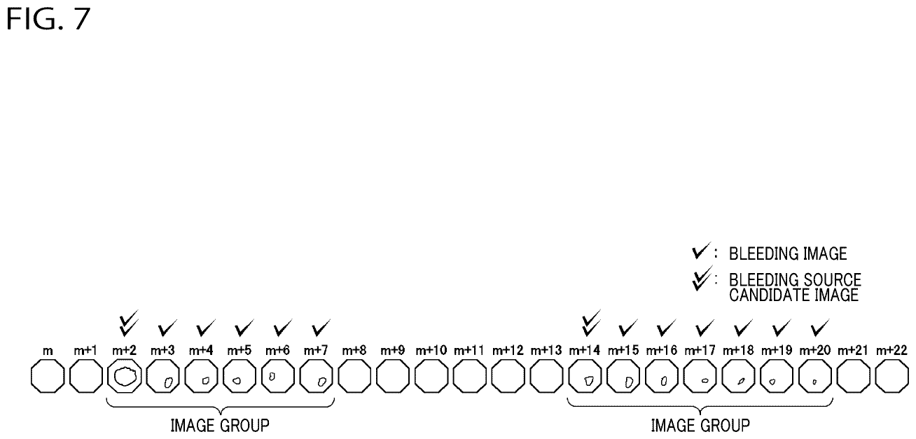

FIG. 7 shows examples of a bleeding source candidate image identified in the multiple endoscopic images shown in FIG. 5. The double check marks shown above the images (m+2) and (m+14) indicate that the images are highly likely to contain a bleeding source, and the bleeding source candidate image identification unit 104 identifies the images (m+2) and (m+14) as the bleeding source candidate images. The bleeding source candidate image identification unit 104 acquires feature values of bleeding images and identifies a bleeding source candidate image based on the feature values thus acquired.

The bleeding source candidate image identification unit 104 may identify a bleeding source candidate image based on at least one of the saturation, hue, or lightness of an area in which blood is shown (hereinafter, also referred to as a “bleeding area”) in a bleeding image. In specific, the bleeding source candidate image identification unit 104 acquires at least one of the saturation, hue, or lightness of bleeding areas and classifies multiple bleeding images into multiple groups based on the results of comparison between at least one of the saturation, hue, or lightness thus acquired and a predetermined criterion. For example, the bleeding source candidate image identification unit 104 may group the multiple bleeding images according to the saturation of a bleeding area having a reddish hue.

For example, when the saturation is expressed in 11 scales from 0 s to 10 s, four groups of saturation ranges may be set as follows.
First group
   Saturation in the range of 9 s to 10 s
Second group
   Saturation in the range of 7 s to 8 s
Third group
   Saturation in the range of 4 s to 6 s
Fourth group
   Saturation in the range of 0 s to 3 s

The bleeding source candidate image identification unit 104 classifies, in the first group, a bleeding image of which the saturation in a bleeding area falls within the range of 9 s to 10 s. A bleeding image classified in the first group contains a bleeding area of which the saturation is too high and the reddish color is too bright. This bleeding area may contain a noise image due to the influence of reflected light or the like.

The bleeding source candidate image identification unit 104 classifies, in the second group, a bleeding image of which the saturation in a bleeding area falls within the range of 7 s to 8 s. A bleeding image classified in the second group contains a bleeding area of which the saturation is high and the redness is strong. It is highly likely that this bleeding area contains a bleeding source.

The bleeding source candidate image identification unit 104 classifies, in the third group, a bleeding image of which the saturation in a bleeding area falls within the range of 4 s to 6 s. A bleeding image classified in the third group contains a bleeding area of which the saturation is to some extent high and the redness is to some extent strong. This bleeding area may contain a bleeding source, which, however, is not likely.

The bleeding source candidate image identification unit 104 classifies, in the fourth group, a bleeding image of which the saturation in a bleeding area falls within the range of 0 s to 3 s. A bleeding image classified in the fourth group contains a bleeding area of which the saturation is low. It is highly unlikely that this bleeding area contains a bleeding source.

As a result of grouping the multiple bleeding images, the bleeding source candidate image identification unit 104 may distinguishably display the bleeding images of multiple groups and may identify a bleeding image classified in the second group as a bleeding source candidate image. Thus, the bleeding source candidate image identification unit 104 may identify a bleeding source candidate image based on at least one of the saturation, hue, or lightness of the bleeding area.

In the following, an example is shown in which the bleeding source candidate image identification unit 104 identifies a bleeding source candidate image based on all of the saturation, hue, and lightness of the bleeding area. For example, when the saturation is expressed in 11 scales from 0 s to 10 s and the lightness is expressed in 17 scales from 1.5 to 9.5, four groups of saturation and lightness ranges may be set as follows.
First group
   Either lightness in the range of 7.5 to 9.5 or saturation in the range of 9 s to 10 s satisfied
Second group
   Lightness in the range of 4.0 to 7.0 and saturation in the range of 7 s to 8 s
Third group
   Lightness in the range of 4.0 to 7.0 and saturation in the range of 4 s to 6 s
Fourth group
   A combination of lightness and saturation that does not meet the conditions of the above three groups

The bleeding source candidate image identification unit 104 classifies, in the first group, a bleeding image of which the lightness in a bleeding area falls within the range of 7.5 to 9.5 or the saturation in the bleeding area falls within the range of 9 s to 10 s. A bleeding image classified in the first group contains a bleeding area of which the lightness and/or saturation is too high and the reddish color is light. This bleeding area often contains a noise image due to the influence of reflected light or the like.

The bleeding source candidate image identification unit 104 classifies, in the second group, a bleeding image of which the lightness in a bleeding area falls within the range of 4.0 to 7.0 and the saturation in the bleeding area falls within the range of 7 s to 8 s. A bleeding image classified in the second group contains a bleeding area of which the saturation is high and the redness is strong. It is highly likely that this bleeding area contains a bleeding source.

The bleeding source candidate image identification unit 104 classifies, in the third group, a bleeding image of which the lightness in a bleeding area falls within the range of 4.0 to 7.0 and the saturation in the bleeding area falls within the range of 4 s to 6 s. A bleeding image classified in the third group contains a bleeding area of which the saturation is to some extent high and the redness is to some extent strong. This bleeding area may contain a bleeding source, which, however, is not likely.

The bleeding source candidate image identification unit 104 classifies, in the fourth group, a bleeding image that does not meet the conditions of the first group through the third group. A bleeding image classified in the fourth group contains a bleeding area of which the lightness and/or saturation is low. It is highly unlikely that this bleeding area contains a bleeding source.

As a result of grouping the multiple bleeding images, the bleeding source candidate image identification unit 104 may distinguishably display the bleeding images of multiple groups and may identify a bleeding image classified in the second group as a bleeding source candidate image. Thus, the bleeding source candidate image identification unit 104 may identify a bleeding source candidate image based on all of the saturation, hue, and lightness of the bleeding area.

Also, the bleeding source candidate image identification unit 104 may derive the percentage occupied by a bleeding area in a bleeding image and identify a bleeding source candidate image based on the percentage.

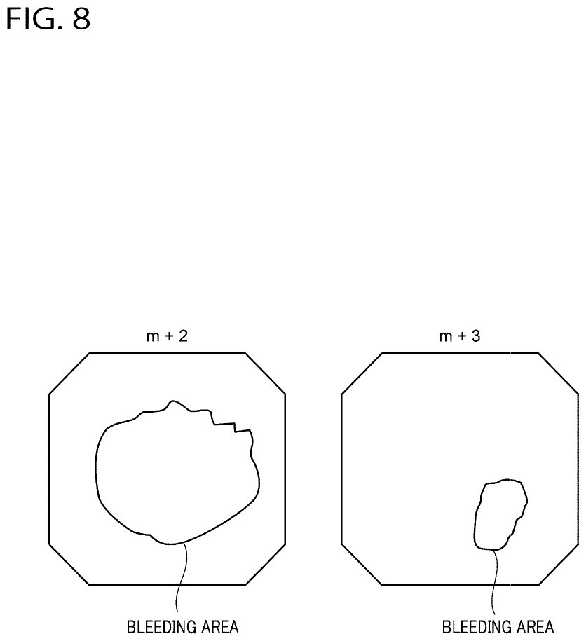

FIG. 8 shows a bleeding area in each of the bleeding images (m+2) and (m+3). Since blood is flowing out (or squirting in some cases) at a bleeding source, the area occupied by the bleeding area including the bleeding source in the entire image is large. Accordingly, the bleeding source candidate image identification unit 104 derives the percentage occupied by a bleeding area in each of bleeding images and classifies the bleeding images into multiple groups based on the results of comparison between the derived percentage and a predetermined criterion. The bleeding source candidate image identification unit 104 may identify, as a bleeding source candidate image, a bleeding image included in a group in which the percentage occupied by the bleeding area is greater than or equal to a predetermined threshold (e.g., 50%) among the multiple groups. In the example shown in FIG. 8, the percentage of the bleeding area in the bleeding image (m+2) is greater than or equal to the threshold, so that the bleeding source candidate image identification unit 104 identifies the bleeding image (m+2) as a bleeding source candidate image.

Also, the bleeding source candidate image identification unit 104 may identify a bleeding source candidate image based on the shooting time of a bleeding image included in an image group. In the case of bleeding in the digestive tract, blood flows from the bleeding source toward the downstream side of the digestive tract. Accordingly, it is presumed that, in an image group including multiple bleeding images, the bleeding source is included in the bleeding image taken on the most upstream side. Therefore, the bleeding source candidate image identification unit 104 refers to the shooting time of each bleeding image included in an image group and identifies, as a bleeding source candidate image, the bleeding image taken on the most upstream side in the image group.

For example, in lower endoscopy, the endoscope 7 inserted to the end of the ileum shoots images while being withdrawn in the direction from the upstream side toward the downstream side in the digestive tract. Accordingly, it is presumed that, among multiple images included in an image group, a bleeding image including a bleeding source was shot at the earliest time. Therefore, the bleeding source candidate image identification unit 104 may identify the bleeding image with the earliest shooting time in an image group in lower endoscopy, as a bleeding source candidate image. In addition to the bleeding image with the earliest shooting time, the bleeding source candidate image identification unit 104 may also identify, as a bleeding source candidate image, a bleeding image that was shot within a predetermined time (e.g., several seconds) from the earliest shooting time. By identifying multiple bleeding source candidate images, a situation can be prevented in which an image that actually contains a bleeding source is not included as a candidate image.

Meanwhile, in upper endoscopy, the endoscope 7 inserted to the duodenum shoots images while being withdrawn in the direction from the downstream side toward the upstream side in the digestive tract. Accordingly, it is presumed that, among multiple images included in an image group, a bleeding image including a bleeding source was shot at the latest time. Therefore, the bleeding source candidate image identification unit 104 may identify the bleeding image with the latest shooting time in an image group in upper endoscopy, as a bleeding source candidate image. In addition to the bleeding image with the latest shooting time, the bleeding source candidate image identification unit 104 may also identify, as a bleeding source candidate image, a bleeding image that was shot between a predetermined time (e.g., several seconds) prior to the latest shooting time and the latest shooting time. By identifying multiple bleeding source candidate images, a situation can be prevented in which an image that actually contains a bleeding source is not included as a candidate image.

In upper endoscopy, when the doctor observes the inside of the digestive tract in the direction from the upstream side toward the downstream side while inserting the endoscope 7, the bleeding source candidate image identification unit 104 may identify the bleeding image with the earliest shooting time in an image group, as a bleeding source candidate image.

When the user selects the bleeding image tab 54d on the report creation screen shown in FIG. 4, the display screen generator 100 generates a list screen for multiple bleeding images including a bleeding source candidate image and displays the list screen on the display device 12b.

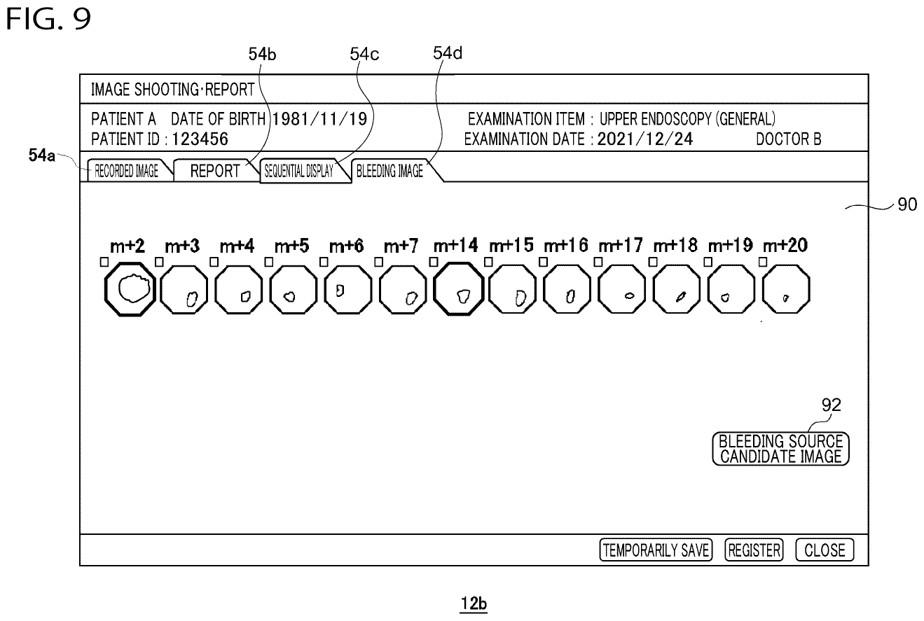

FIG. 9 shows an example of a list screen 90 for bleeding images. In the list screen 90, endoscopic images identified as bleeding images are arranged according to the order of shooting. The endoscopic images may be displayed as reduced thumbnail images. The display screen generator 100 may refer to the additional information of each endoscopic image and display, with the endoscopic image, the image ID of the endoscopic image, and a region name indicating a region included in the endoscopic image.

The display screen generator 100 displays a bleeding image identified as a bleeding source candidate image in a different manner from a bleeding image not identified as a bleeding source candidate image. In the example shown in FIG. 9, the display screen generator 100 displays the outer frame of each bleeding source candidate image in a different color or with a different thickness from the outer frame of each of the other bleeding images so that the bleeding source candidate images can be distinguished from the other bleeding images. The display screen generator 100 may also add a mark around each bleeding source candidate image, for example, so that the bleeding source candidate image can be distinguished from the other bleeding images. In this example, the images (m+2) and (m+14) are displayed in a different manner from the other bleeding images so that the user can easily recognize them as the bleeding source candidate images.

For each endoscopic image displayed on the list screen 90, a checkbox is provided. When the user operates the mouse to place the mouse pointer in a checkbox and left-clicks, the operation receiver 82 accepts it as an operation to select the endoscopic image as an attached image of the report, so that the endoscopic image is selected as an attached image of the report. When the report creation screen is displayed, the endoscopic images selected as the attached images of the report are arranged and displayed in the attached image display area 56 (see FIG. 4).

When the user operates a switch button 92 on the list screen 90, the display screen generator 100 generates a list screen for bleeding source candidate images and displays the list screen on the display device 12b.

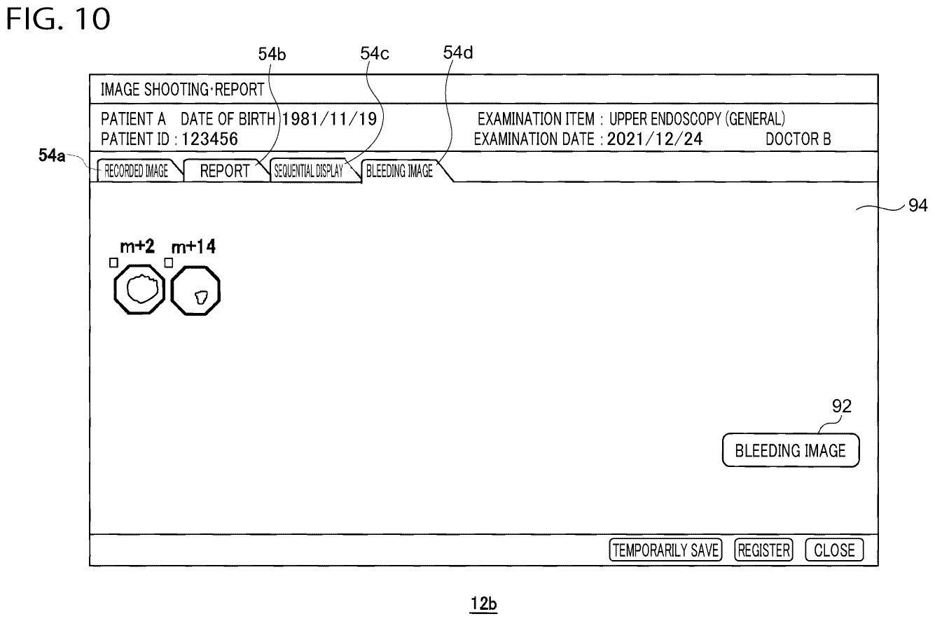

FIG. 10 shows an example of a list screen 94 for bleeding source candidate images. In the list screen 94, endoscopic images identified as bleeding source candidate images are arranged according to the order of shooting. A bleeding source candidate image is an image that is highly likely to contain a bleeding source, and, since the user can observe only the bleeding source candidate images carefully, the user can identify the bleeding source promptly.

In the embodiment, the bleeding source candidate image identification unit 104 classifies multiple bleeding images into four groups. In the example described above, the bleeding source candidate image identification unit 104 automatically identifies the bleeding images classified in the second group as bleeding source candidate images. Meanwhile, in the following example, when the user operates the input unit 78 to select one of the four groups, the operation receiver 82 acquires the group selection operation, and the bleeding source candidate image identification unit 104 identifies the endoscopic images classified in the selected group as bleeding source candidate images.

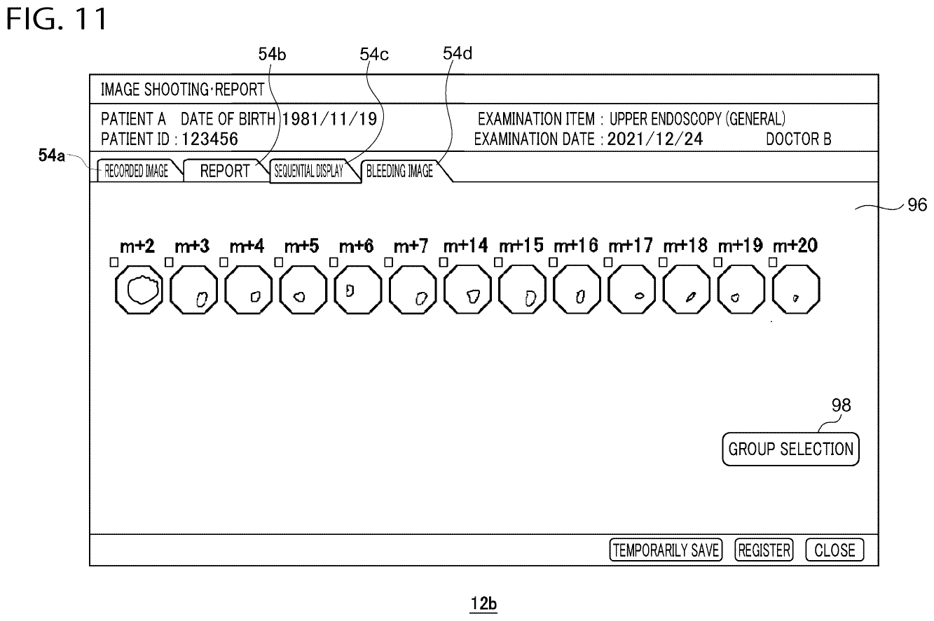

FIG. 11 shows another example of a list screen 96 for bleeding images. In the list screen 96, endoscopic images identified as bleeding images are arranged according to the order of shooting. At this point, no bleeding source candidate image has been identified. When the user operates a group selection button 98, the display screen generator 100 generates a group selection screen and displays it on the display device 12b.

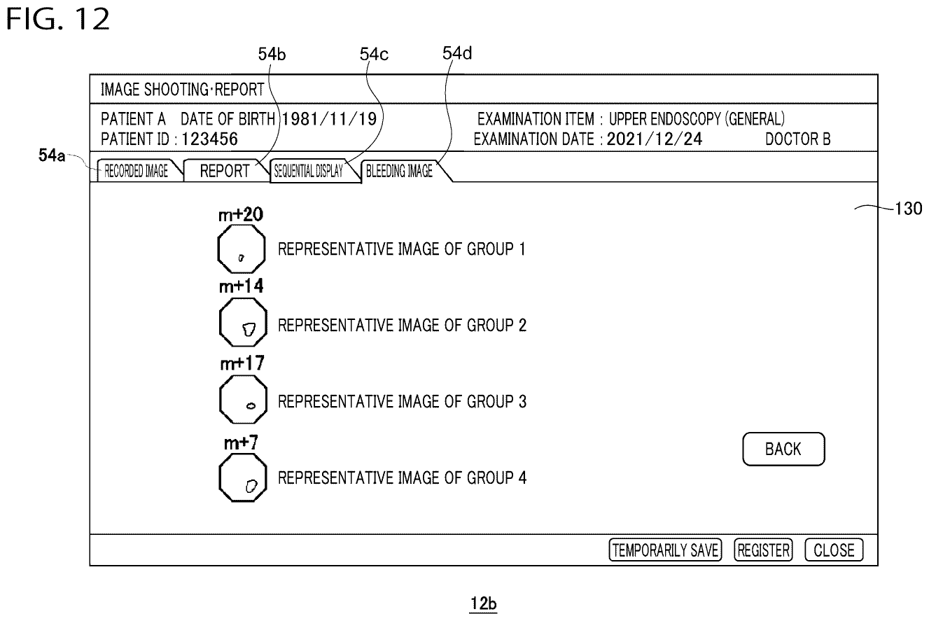

FIG. 12 shows an example of a group selection screen 130. The display screen generator 100 distinguishably displays bleeding images belonging to each group on the group selection screen 130. In the embodiment, the display screen generator 100 displays the image (m+20) as a representative image of a group 1, displays the image (m+14) as a representative image of a group 2, displays the image (m+17) as a representative image of a group 3, and displays the image (m+7) as a representative image of a group 4.

The representative image of each group is selected from each group by the bleeding source candidate image identification unit 104. The bleeding source candidate image identification unit 104 may identify, as a representative image in each group, a bleeding image with the highest saturation in the bleeding area, a bleeding image with the highest lightness in the bleeding area, or a bleeding image with the largest area of the bleeding area. The bleeding source candidate image identification unit 104 may also identify, as a representative image in each group, a bleeding image of which the saturation in the bleeding area shows an average value, a bleeding image of which the lightness in the bleeding area shows an average value, or a bleeding image of which the area of the bleeding area shows an average value. The bleeding source candidate image identification unit 104 may assign information indicating the probability that a bleeding image contains a bleeding source to each of the multiple groups, and the display screen generator 100 may display the representative image of each group together with the information indicating the probability. This probability information may be used by the user to select a group.

On the group selection screen, the user views the representative image of each group and selects a group that is presumed to include a bleeding source. The user may be able to select multiple groups. When the user selects the representative image of a group using the mouse, the operation receiver 82 acquires the group selection operation. The bleeding source candidate image identification unit 104 identifies a bleeding image belonging to the selected group as a bleeding source candidate image, and the display screen generator 100 displays a bleeding image belonging to the group selected by the user as a bleeding source candidate image. For example, when the user has selected the group 3, the display screen generator 100 generates a list screen in which the bleeding images belonging to the group 3 are arranged and displays the list screen on the display device 12b.

At the time, the display screen generator 100 may display the multiple bleeding images belonging to the group selected by the user, in descending order of red saturation. With such display, the user can focus on a bleeding source candidate image that is highly likely to contain a bleeding source. The display screen generator 100 may also display the multiple bleeding images belonging to the group selected by the user, in descending order of lightness.

When the display screen generator 100 displays multiple bleeding images (a first bleeding image and a second bleeding image, for example) belonging to the group selected by the user, the image group identification unit 106 judges whether the first bleeding image and the second bleeding image were shot in the same bleeding section or in different bleeding sections. When the image group identification unit 106 has already identified multiple image groups, the bleeding sections of the first bleeding image and the second bleeding image may be identified by investigating whether the first bleeding image and the second bleeding image are included in the same image group or in different image groups.

When the image groups have not been identified, the image group identification unit 106 judges whether the first bleeding image and the second bleeding image were shot in the same bleeding section or in different bleeding sections, based on at least one of the distance between the shooting positions of the first bleeding image and the second bleeding image, the interval between the shooting times of the first bleeding image and the second bleeding image, or the number of other images shot between the shooting of the first bleeding image and the shooting of the second bleeding image. When the first bleeding image and the second bleeding image were shot in different bleeding sections, the display screen generator 100 may display the first bleeding image and the second bleeding image in different manners. This makes the user aware that bleeding source candidate images in different bleeding sections are displayed.

When the user selects the sequential display tab 54c on the report creation screen shown in FIG. 4, the display screen generator 100 generates a playback screen on which multiple endoscopic images taken inside a subject's body are sequentially displayed in chronological order of shooting and displays the playback screen on the display device 12b.

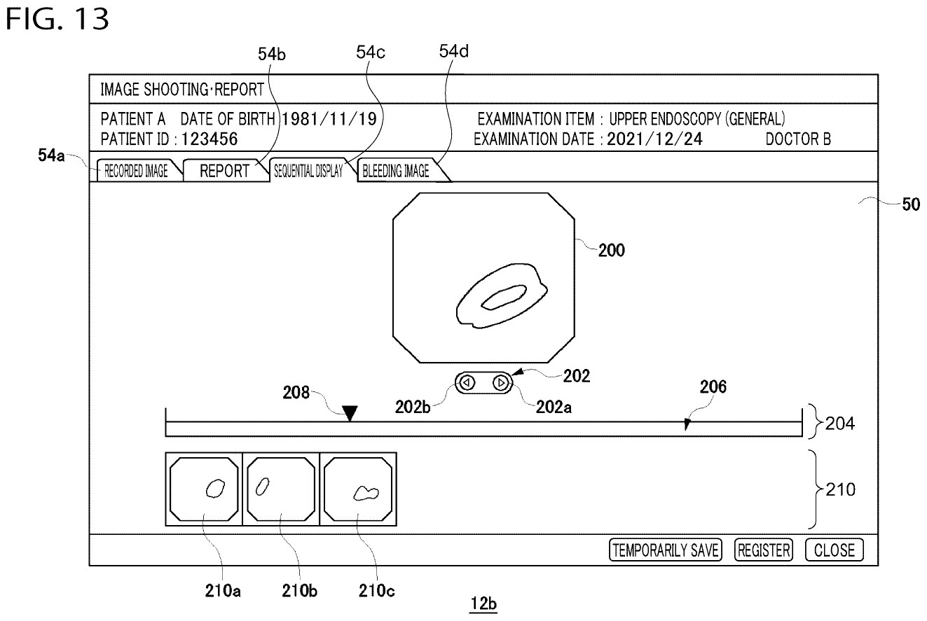

FIG. 13 shows an example of a playback screen 50 for endoscopic images. In an upper center part of the playback screen, a playback area 200 is provided in which multiple endoscopic images are sequentially displayed while being switched therebetween. In a playback button display area 202, a playback button 202a and a reverse playback button 202b are displayed. When the playback button 202a is selected, endoscopic images are sequentially displayed in the forward direction (the direction from the image with the earliest shooting time to the image with the latest shooting time) in the playback area 200. When the reverse playback button 202b is selected, endoscopic images are sequentially displayed in the reverse direction (the direction from the image with the latest shooting time to the image with the earliest shooting time) in the playback area 200.

When the playback button 202a or the reverse playback button 202b is selected, the display controller 110 displays multiple endoscopic images in sequence in the playback area 200 while switching between the endoscopic images. At the time, in place of the playback button 202a or reverse playback button 202b selected, a pause button is displayed. When the user operates the pause button during the sequential display of endoscopic images, the display controller 110 pauses the sequential display of the endoscopic images and displays a still image of the endoscopic image that was displayed at the time when the pause button was operated.

When the user places the mouse pointer on the image displayed in the playback area 200 and double-clicks the left mouse button, the image is selected as an attached image and displayed in an attached image display area 210. In this example, it is shown that three attached images 210a-210c have been selected.

The display screen generator 100 displays, below the playback area 200, a bar display area 204 of horizontally long shape with a shooting start time at one end and a shooting end time at the other end. The bar display area 204 of the embodiment represents a time axis with the shooting start time at the left end and the shooting end time at the right end. The bar display area 204 may also indicate the shooting order of the images, with the image with the earliest shooting time arranged at the left end and the image with the latest shooting time arranged at the right end. A slider 208 indicates a temporal position of the endoscopic image displayed in the playback area 200. When the user places the mouse pointer on an arbitrary location in the bar display area 204 and clicks the left mouse button, the endoscopic image located at the time position is displayed in the playback area 200. Also when the user drags the slider 208 and drops it at an arbitrary position in the bar display area 204, the endoscopic image located at the time position is displayed in the playback area 200.

The display controller 110 displays, in the bar display area 204, a color bar 206 of band shape that indicates a temporal change in the color information of shot endoscopic images. The color bar 206 is configured by arranging, in chronological order, the color information of multiple endoscopic images acquired in the examination.

The display controller 110 has a function to, when the user selects one image on this playback screen 50, sequentially display images, starting from the one image to a bleeding point candidate image, automatically in the reverse direction or the forward direction. The direction of the automatic playback depends on the direction of observation in the endoscopic examination. When image shooting is performed in the direction from the upstream side toward downstream side of the digestive tract (e.g., lower endoscopy), the automatic playback direction is set to the reverse direction; when image shooting is performed in the direction from the downstream side toward upstream side of the digestive tract (e.g., upper endoscopy), the automatic playback direction is set to the forward direction.

When the operation receiver 82 acquires, from the user, an image selection operation for automatic sequential display, the display controller 110 automatically displays images from the selected image to a bleeding source candidate image in sequence. This function allows the user to carefully observe images near the bleeding source candidate image.

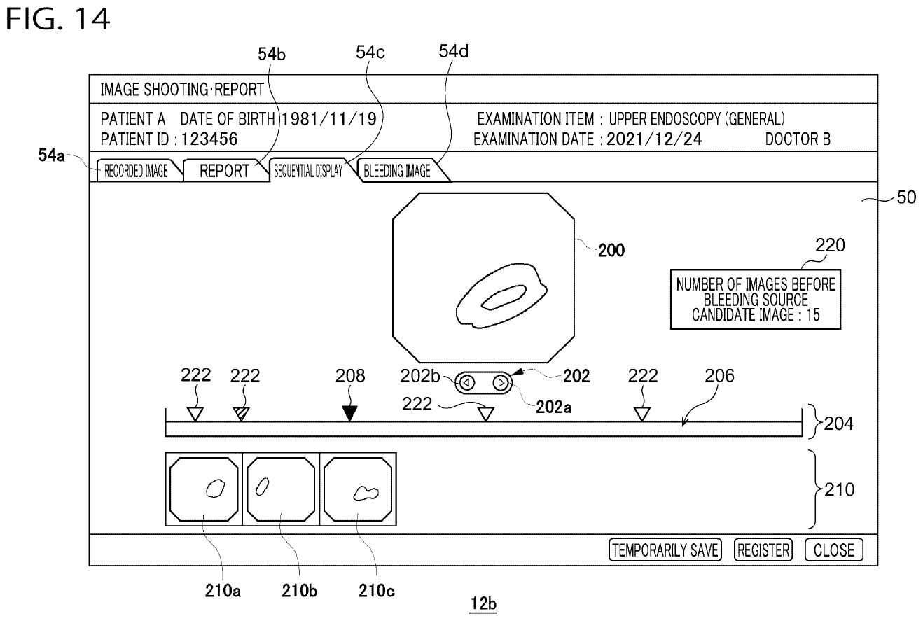

FIG. 14 shows an example of a screen displayed when the user has selected an image for automatic sequential display. In an automatic playback mode, the display controller 110 acquires the number of images from the selected image to a bleeding source candidate image and displays, on the playback screen 50, notification information 220 indicating the number of the images. By viewing the playback screen 50, the user can check the timing at which the bleeding source candidate image is displayed. The display controller 110 may acquire the time required to display the images from the selected image to the bleeding source candidate image (the time required until the bleeding source candidate image is displayed) and display the notification information 220 indicating the time required for the image display. Also, both the number of images and the time required until the bleeding source candidate image is displayed may be displayed as the notification information 220.

The display controller 110 may update the notification information 220 during the sequential display of images. More specifically, when one image is displayed in the playback area 200, the display controller 110 reduces the “number of images before a bleeding source candidate image” by 1 or reduces the “time until a bleeding source candidate image is displayed” by the display time of one image. In this way, the display controller 110 may acquire the number of images between the image currently displayed and a bleeding source candidate image or the time required until a bleeding source candidate image is displayed and may display the number of images or the time required for the image display thus acquired, as the notification information 220 on the playback screen 50.

When displaying a bleeding source candidate image in the playback area 200, the display controller 110 stops the sequential display, displays the bleeding source candidate image as a still image, and may display the bleeding source candidate image in a predetermined display manner to notify the user that a bleeding source candidate image is displayed. For example, the display controller 110 may blink the frame of the bleeding source candidate image, may change the color of the frame, or may add a mark around the bleeding source candidate image.

When there is another bleeding source candidate image nearby other than the bleeding source candidate image being displayed as a still image, the display controller 110 may notify the user thereof. For example, when multiple bleeding source candidate images have been identified in the same bleeding section, each of the bleeding source candidate images may contain the bleeding source. Therefore, by allowing the display controller 110 to notify the user of the presence of another bleeding source candidate image, an occasion to perform further automatic sequential display can be provided to the user.

During the automatic sequential display, the display controller 110 moves the slider 208 according to the current playback position. At the time, the display controller 110 may place, in the bar display area 204, marks 222 that respectively indicate the positions of multiple bleeding source candidate images, so as to make the user aware of the positional relationships between the current playback position and the bleeding source candidate images. During the automatic sequential display, the display controller 110 may display the mark 222 for the bleeding source candidate image closest to the playback position in the playback direction, in a different manner from the marks 222 for the other bleeding source candidate images. When displaying a bleeding source candidate image in the playback area 200 during the automatic sequential display, the display controller 110 displays the mark 222 for the bleeding source candidate image in a normal manner and displays the mark 222 for the bleeding source candidate image to be displayed next in a different manner from the normal manner. This can make the user easily aware of the positional relationship between the current playback position and the bleeding source candidate image to be displayed next.

In the report creation operation, the user selects an image to be attached to the report and enters an examination result into the entry area 58 in the report creation screen to create the report. When the user operates a registration button (see FIG. 4), the registration processing unit 112 registers, in the server device 2, the information entered into the report creation screen, and the report creation operation is completed.

The present disclosure has been described based on an embodiment. The embodiment is intended to be illustrative only, and it will be obvious to those skilled in the art that various modifications to a combination of constituting elements or processes could be developed and that such modifications also fall within the scope of the present disclosure. In the embodiment, the display screen generator 100 arranges and displays bleeding images according to the order of shooting. However, the bleeding images may be arranged and displayed in descending order of the percentage occupied by a bleeding area, for example. Since a bleeding image with a large percentage occupied by a bleeding area is highly likely to contain a bleeding source, such display allows the user to efficiently identify an image that contains a bleeding source.

During the automatic sequential display, the display controller 110 displays all endoscopic images taken in an endoscopic examination. However, only bleeding images may be displayed, for example. Also, when endoscopic images taken in an endoscopic examination have been compressed in different methods, only the images compressed in a format with high image quality may be displayed. For example, when there are inter-frame compressed images and intra-frame compressed images, only the intra-frame compressed images with higher image quality may be displayed.

In the embodiment, the endoscope observation device 5 transmits a capture image to the image storage device 8. However, in a modification, the image analysis device 3 may transmit a capture image to the image storage device 8. Also, although the information processing device 11b includes the processing unit 80 in the embodiment, the server device 2 may include the processing unit 80 in a modification.

The embodiment describes a method for efficiently displaying multiple bleeding images acquired by a doctor using the endoscope 7 inserted into a patient's digestive tract. The subject method is applicable when multiple bleeding images acquired by a capsule endoscope with a shooting frame rate higher than 2 fps are displayed. For example, when the shooting frame rate is 8 fps and if the image shooting of the inside of the body is performed for about 8 hours, about 230,000 images of the inside of the body will be acquired. Since the number of images acquired in capsule endoscopy is enormous, the subject method can be effectively applied.

Also, in the embodiment, a situation is assumed in which the user observes a bleeding image after the examination is finished. However, during the examination, for example, information regarding a bleeding source candidate image may be provided to the user.

What is claimed is:

1. A medical assistance system comprising a processor including hardware,
the processor is configured to:
classify a plurality of images taken inside a subject's body into a plurality of groups based on a predetermined criterion and
display the plurality of groups distinguishably from each other.

2. The medical assistance system according to claim 1, wherein the processor is configured to:
acquire at least one of saturation, hue, or lightness of an area in which bleeding is shown in the image and
classify the plurality of images into the plurality of groups based on a result of comparison between at least one of the saturation, the hue, or the lightness acquired and the predetermined criterion.

3. The medical assistance system according to claim 1, wherein the processor is configured to:
assign information indicating the probability that the image contains a bleeding source, to each of the plurality of groups and
display the image belonging to each of the plurality of groups, together with the information indicating the probability.

4. The medical assistance system according to claim 1, wherein the processor is configured to:
acquire a selection operation for the group from a user and
display the image belonging to the group selected by the selection operation, as a bleeding source candidate image that is likely to contain a bleeding source where blood is flowing out.

5. The medical assistance system according to claim 4, wherein the processor is configured to:
display the images belonging to the group selected by the selection operation, in descending order of red saturation or in descending order of lightness.

6. The medical assistance system according to claim 1, wherein the processor is configured to:
judge whether a first image and a second image classified in the same group were shot in the same bleeding section or shot in different bleeding sections and
display the first image and the second image in different manners when the first image and the second image were shot in different bleeding sections.

7. The medical assistance system according to claim 6, wherein the processor is configured to:
judge whether the first image and the second image were shot in the same bleeding section or shot in different bleeding sections, based on at least one of the distance between shooting positions of the first image and the second image, the interval between shooting times of the first image and the second image, or the number of other images shot between shooting of the first image and shooting of the second image.

8. The medical assistance system according to claim 1, wherein the processor is configured to:
identify, as a representative image in each of the groups, the image with the highest saturation in an area in which blood is shown, the image with the highest lightness in the area, or the image with the largest area of the area and
display the representative image of each of the plurality of groups.

9. The medical assistance system according to claim 1, wherein the processor is configured to:
derive the percentage occupied by an area in which blood is shown in the image and
classify the plurality of images into the plurality of groups based on a result of comparison between the percentage derived and the predetermined criterion.

10. The medical assistance system according to claim 9, wherein the processor is configured to:
display the image included in the group in which the percentage is greater than or equal to a predetermined threshold among the plurality of groups, as a bleeding source candidate image that is likely to contain a bleeding source where blood is flowing out.

11. The medical assistance system according to claim 1, wherein the processor is configured to:
classify the plurality of images into the plurality of groups based on the shooting time of each of the images.

12. The medical assistance system according to claim 11, wherein the processor is configured to:
classify the plurality of images into the plurality of groups based on at least one of the distance between shooting positions of two of the images, the interval between shooting times of two of the images, or the number of other images shot between shooting operations for two of the images.

13. The medical assistance system according to claim 9, wherein the processor is configured to:
display the images in descending order of the percentage of the area.

14. The medical assistance system according to claim 1, wherein the processor is configured to:
acquire a selection operation for the image from a user and
display image in sequence from a selected image selected by the selection operation to a bleeding source candidate image that is likely to contain a bleeding source where blood is flowing out.

15. The medical assistance system according to claim 14, wherein the processor is configured to:
acquire the number of images between the selected image and the bleeding source candidate image, or the time required until the bleeding source candidate image is displayed and
display the number of images acquired or the time required for the image display acquired.

16. The medical assistance system according to claim 14, wherein the processor is configured to:
acquire the number of images between the image being displayed and the bleeding source candidate image, or the time required until the bleeding source candidate image is displayed and
display the number of images acquired or the time required for the image display acquired.

17. The medical assistance system according to claim 14, wherein the processor is configured to:
perform predetermined notification when the bleeding source candidate image is displayed.

18. An image display method, comprising:
classifying a plurality of images taken inside a subject's body into a plurality of groups based on a predetermined criterion; and
displaying the plurality of groups distinguishably from each other.
